(19) United States
(12) Patent Application Publication
(10) Pub. No.: US 2003/0027133 A1
(43) Pub. Date: Feb. 6, 2003

(54) Method of improving a mixture experiment

(76) Inventor: James Norman Cawse, Pittsfield, MA (US)

Correspondence Address: GENERAL ELECTRIC COMPANY, GLOBAL RESEARCH CENTER, PATENT DOCKET RM. 4A59, PO BOX 8, BLDG. K-1 ROSS, NISKAYUNA, NY, 12309, US

(21) Appl. No.: 09/681,751

(22) Filed: May 31, 2001

(51) Int. Cl. C12Q 1/68; G01N 33/53; G06F 19/00; G01N 33/48; G01N 33/50; G01N 33/543

(52) U.S. Cl. 435/6; 435/7.1; 436/518; 702/19

(57) ABSTRACT

An array of a mixture of at least two components is formed and an experiment is conducted on the array to produce results. At least one Six Sigma technique is applied to the steps to improve results of the experiment.

### BACKGROUND OF INVENTION

The present invention relates to a method for improving a mixture experiment. Mixture experiments relate to the testing of multicomponent gradient combinations. In a mixture experiment, response is assumed to depend only on relative proportions of the ingredients or factors present in the mixture and not on the amount of the mixture. In a mixture experiment, if the total amount is held constant and the value of the response changes when changes are made in the relative proportions of the ingredients or factor levels making up the mixture, then the behavior of the response is said to be a measure of the joint blending property of the ingredients or factors in the mixture. John A. Cornell, Experiments with Mixtures, 2^{nd} Ed., p. 13, 1990.

One type of mixture experiment involves preparation of a gradient array. For example, the development of materials such as phosphors for lighting applications can involve the testing of gradient arrays of materials by a methodology called combinatorial high throughput screening (CHTS). Sun, Combinatorial Search for Advanced Luminescence Materials, Biotechnology and Bioengineering (Combinatorial Chemistry), vol. 61, 4, pp. 193, 201 (1999). The methodology of CHTS as applied to materials evolved from combinatorial organic synthesis (COS). COS is a high throughput screening (HTS) method that uses systematic and repetitive synthesis to produce libraries of diverse pharmaceutical molecular entities formed from sets of chemical “building blocks.” COS applies automation and miniaturization to produce the libraries through successive stages, each of which produces a chemical modification of an existing molecule of a preceding stage. For example, Pirrung et al., U.S. Pat. 5,143,854 discloses a technique for generating arrays of peptides and other molecules using light-directed, spatially-addressable synthesis techniques.

If properly conducted, a CHTS gradient method can be used to predict successful commercial applications. However, because of the complexity of the systems investigated by CHTS gradient methods, small error in the steps of the method can produce erroneous results that incorrectly predict commercial application. Vast sums of money may be invested into scaling the results of a CHTS gradient investigation to a commercial application. Hence, the effect of small error in the investigatory method can lead to commercial disaster. Currently, no standards or methods exist to monitor CHTS accuracy. There is a need for a methodology to investigate and measure error in this area.

### SUMMARY OF INVENTION

The invention meets this need by providing a method to investigate and measure error and to improve mixture experiments, particularly CHTS mixture experiments. In the method, an array of a mixture of at least two components is formed and an experiment is conducted on the array to produce results. At least one Six Sigma technique is applied to the steps to improve results of the experiment.

In an embodiment of the invention, a method is provided wherein a reactant delivering step is identified as an opportunity for a defect in a combinatorial high throughput screening, a number of units produced by the delivering step is measured, defects in the units produced by the delivering step of the repeated CHTS are measured and defects per unit is calculated for the delivering step.

In a final embodiment, a method is provided a reactant delivering step or stock formulating step is identified as an opportunity for a defect in a mixture experiment, a number of units produced by the delivering step or formulating step is measured, defects in the units produced by the delivering step or the formulating step are measured and a defects per unit is measured for the delivering step or formulating step.

### BRIEF DESCRIPTION OF DRAWINGS

FIG. 1 is a schematic representation of a CHTS dispenser assembly;

FIG. 2 is an overall method for conducting c CHTS experiment;

FIGS. 3 and 4 are graphic representations of ternary mixture experiments;

FIGS. 5A to 5D are histographs of results;

FIG. 6 is a graphic representation of a ternary mixture experiment; and

FIGS. 7A and 7B are histographs of results.

### DETAILED DESCRIPTION

Quality is an important issue for manufacturers. When manufactured products having defects are produced and sold, the result is lost manufacturing time as well as unfavorable publicity. Reduction in error during a CHTS experiment to develop a manufactured materials product has been found to significantly contribute to total quality management of a manufactured product. In one embodiment, the invention relates to a methodology to examine a CHTS experiment particularly a gradient mixture CHTS experiment, to identify a critical to quality defect opportunity (CTQ), to apply a statistical analysis of the CTQ and to monitor the experiment to determine CTQ improvement.

Analysis to improve the experiment is based on a Sigma value. Sigma value is a metric of the Six Sigma program to reduce defects within products. The Six Sigma program provides various statistical tests that workers and managers can use to measure the quality of products, both in development and in production. The Sigma value is an expression of variation from specification or an expression of a frequency of defects with respect to a total number of opportunities. The advantage of the Sigma value is that it is an absolute value that can be compared to quality evaluations of many disparate processes. For example, once a Sigma value is determined for say a product of a method of manufacturing, the value can be compared on a Sigma scale basis to any other process product, say a product of a method of marketing. Additionally, the Sigma value provides a comparison metric to determine improvement in the processes.

A Sigma value can be derived from at least one measurement of quality: a ratio of the variability of a system to specifications on the system or a count of defects compared to a number of opportunities for defects. In the first case, a Sigma value can be determined by the formula (I):

$$Sigma = \frac{\begin{matrix}\vert \text{average value of measurements on the system} - \\ \text{nearest specification} \vert\end{matrix}}{\text{standard deviation of the measurements on the system}} \qquad ( I )$$

(The vertical bars μ indicate the absolute value function.)

In the second case, the Sigma value is per million occurrences of the ratio of number of defects of a product to number of opportunities times the number of units. Eckes, The Six Sigma Revolution 99, (2001). A defect is a variation in a characteristic of a product that is far enough removed from a target value so as to prevent the product from functioning properly. An “opportunity” is anything that must be correct to produce a defect-free product or service, or alternatively, anything that might go wrong to keep a product from working. In summary, an opportunity is a chance for a defect to occur. For example, opportunities can be steps in a manufacturing process, or parts and leads on a circuit card assembly. “Defects per unit” (DPU) is an average number of defects per unit. The DPU can be normalized to one million opportunities (number of defects per million opportunities, DPMO) according to the following:

DPMO=[(defects)/(units)(opportunities)]×10⁶ (II)

DPMO is related to the area under the tail of a standard normal curve at (Sigma-1.5) standard deviations out from the center of the curve. Table 1 summarizes the relationship between DPMO and Sigma.

TABLE 1

| Sigma | DPMO |
|---|---|
| 2.00 | 309000 |
| 2.25 | 227000 |
| 2.50 | 159000 |
| 2.75 | 106000 |
| 3.00 | 66800 |
| 3.25 | 40100 |
| 3.50 | 22800 |
| 3.75 | 12200 |
| 4.00 | 6210 |
| 4.25 | 29.80 |
| 4.50 | 1350 |
| 4.75 | 580 |
| 5.00 | 233 |
| 5.25 | 88 |
| 5.50 | 32 |
| 5.75 | 11 |
| 6.00 | 3.4 |

In one embodiment, the invention relates to a CHTS method comprising (A) an iteration of steps of (i) formulating an array of mixtures of at least two components; (ii) reacting the array mixtures; and (iii) evaluating a set of products of the reacting step and (B) repeating the iteration of steps (i), (ii) and (iii) wherein components of a successive array of mixtures selected for a step (i) are chosen as a result of an evaluating step (iii) of a preceding iteration.

These and other features will become apparent from the drawings and following detailed discussion, which by way of example without limitation describe preferred embodiments of the invention.

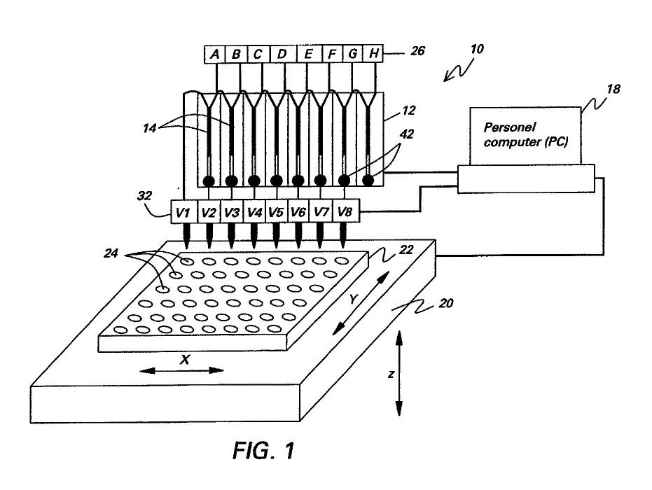

FIG. 1 schematically represents a combinatorial high throughput screening dispensing assembly 10 with an array of 8 positive displacement syringes. Assembly 10 includes a battery 12 of syringes 14 that is driven by stepping motor (not shown), which in turn is controlled by computer 18. The dispensing assembly 10 further includes X-Y-Z robotic positioning stage 20, which supports array plate 22. X-Y-Z robotic positioning stage 20 is controlled by computer 18 to position wells 24 of the array plate 22 beneath respective syringes 14 for delivery of test solutions from reservoirs 26.

Computer 18 controls aspiration of precursor solution into the battery 12 of syringes 14 and sequential positioning of the wells 24 of array place 22 so that a prescribed stoichiometry and/or composition of precursor can be delivered to the wells 24. By coordinating activation of the syringes 14 and movement of plate 22 on the robotic X-Y-Z table 20, reactants can be generated in a two-dimensional array for use in a combinatorial high throughput screening method. The array of reactants is part of a CHTS library. A library is a physical, trackable collection of samples that can be subjected to a definable set of processes or reaction steps and screened for various activities.

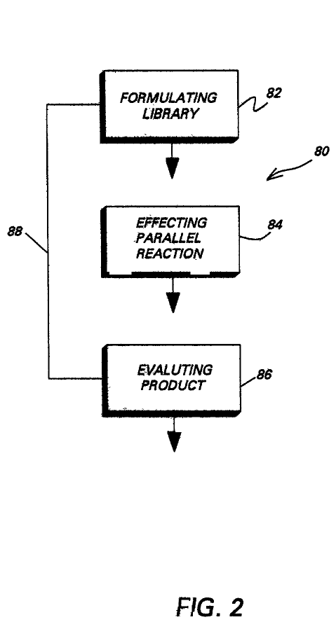

In one embodiment, the CHTS can be described with reference to FIG. 2 as a method 80 comprising (A) (i) formulating 82 a library of reactants by dispensing a solution of the precursor into a well of an array plate; (ii) effecting 84 a reaction of the precursor to produce product; and (iii) evaluating 86 the product. The method includes (B) reiterating 88 (A) wherein a successive library for a step (i) is selected for formulating as a result of an evaluating step (iii) 86 of a preceding iteration of (A).

In accordance with the invention, at least one Six Sigma technique is applied to a step of the experiment to improve results. A first step of a Six Sigma technique can comprise identifying a defect opportunity in the experiment. A defect opportunity can include any step of monitoring a stock precursor solution, mixing an aliquot of the stock solution with an aliquot of another solution, delivering a mixture of the aliquots to a well of an array plate, effecting a condition of reaction on the mixture, detecting a result of the reaction and analyzing the result to determine either a lead or to determine a candidate library for reiterating the experiment.

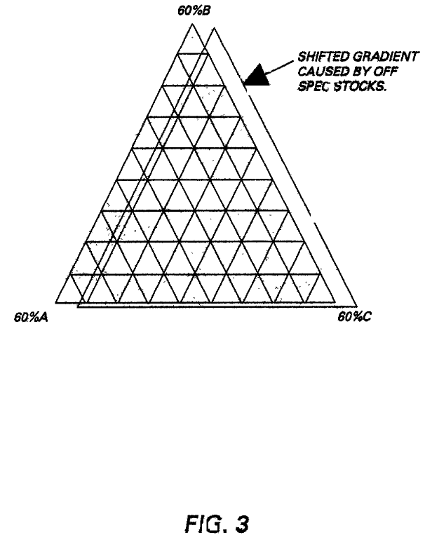

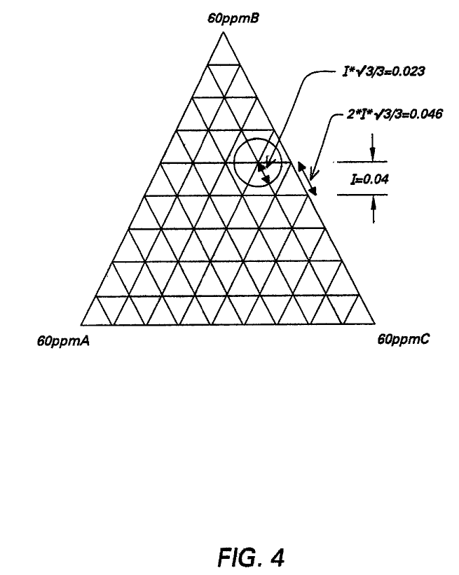

Critical to quality (CTQ) defect areas are identified within the defect opportunities. For example, it has been found that the step of formulating or delivering stock solution can be a CTQ area. As shown in FIG. 3, defects in this step will substantially shift a ternary gradient experimental space. The FIG. 3 shows a typical ternary gradient in which the components A, B, and C each have ranges from low=20% to high=60%. Thus at the vertex labeled 60% A, the mixture composition is 60% A, 20% B, and 20% C. The gradient is measured in 11 total intervals M from a lowest to a highest level for each component. Each interval I is then equal to (1/(M−1))(high to low). In FIG. 4, expressed in percentage terms, I=(1/(11−1))*(60−20)=4%.

Each intersection point in FIG. 4 specifies a sample to be made and measured. The sample points form equilateral triangles of height 1. Hence, distance between adjacent points is (2*I/3*√3). If a delivered concentration of components at a point differs from designed values (those specified by the experimental plan, with no error included) by more than 2 that distance, or (I/3*√3), the results from the determination of properties at that point will effectively be those from an adjacent point. The resulting confusion in results can be defined as a defect in the Six Sigma process. Accordingly, a Six Sigma specification for each point in the gradient can be specified as an actual concentration that deviates no more than (I/3*√3) from the design concentration (those specified by the experimental plan, with no error included). The equation for as-delivered concentration of a given component can be derived and represented according to the following definitions and formulas:

Design concentration of the stock solution of component i: Sᵢ

Design amount of stock solution of component i added to mixture: Aᵢ

Variance in the concentration of stock solution of component i: σ²

Concentration fraction of component 1 in the gradient:

G₁=A₁S₁/ΣAᵢSᵢ (III)

Variance of component 1 (σ²^{G1}) can be represented:

$${σ}_{G1}^{2} = {\sum }_{i} [ \frac{\partial {G}_{1}}{\partial {S}_{1}} ] {σ}_{{S}_{i}}^{2} \qquad ( IV )$$

The variance of other components (σ²_{G2}, σ²_{G3}, . . . ) can be represented similarly, e.g. by changing the subscript on G from 1 to 2 for component 2.

Total variance around a given point in the gradient (σ²_{P}):

σ²_{P}=σ²_{G1}+G²_{G2}+σ²_{G3}+ . . . (V)

and the standard deviation around the point (σ_{P}) is

σ_{P}=√σ²_{P} (VI)

The total variance around the point represents a performance metric that can be transformed to a Sigma scale of measure. A Sigma value can be assigned to a quality of hitting the various concentration points according to the following which is derived from the formula (I) for Sigma, where the average value=0, the specification=I/3*√3, and the standard deviation=σₚ

Sigma=(I/3*√3)σ_{P} (VII)

Quality goals of a particular program can be specified by a Sigma value. In the experiment described in this application, project goal Sigma values can be at least 4.5, desirably at least 5.0 and preferably at least 5.5. The actual value of Sigma can be determined as a ratio of the variability of a system to specifications on the system. This process is exemplified by the following procedure for a ternary system with intervals I:

1. A point on a gradient is selected at random. For example, (G1,G2, G3)=(0.32,0.32,0.36).

2. A design concentration for each stock solution (S₁ . . . S₃) and an estimate of the standard deviation for each stock solution (σ₁ . . . σ₃) iS selected.

3. A n amount (A₁ . . . A₃) of each stock required to generate a concentration fraction (G₁,G₂,G₃) of the point mixture is determined according to formulas (III) through (VII) based on an assumption of no error in stock solution concentration.

4. Va lues of delivery stock concentrations S″₁ . . . S″₃ are randomly selected from normal distributions having mean S₁ . . . S₃ and standard deviations σ₁ . . . σ₃

5. Delivered concentrations (G₁″ . . . G₃″) of the components of the mixture resulting from mixing quantities (A₁ . . . A₃) of stock solutions (S₁″ . . . S₃″) are calculated.

6. Distances between delivered and design concentrations of components is calculated according to the formula (where SQRT is the square root function):

Distance=SQRT((G₁″−G₁)ˆ ₂+(G₂′−G₂)ˆ 2+(G₃′−G₃)) (VIII)

7. The distance between a delivered concentration and design concentration is compared with the value I/3*√3. A defect is counted when a distance is greater than I/3*√3.

8. Ste ps 4-7 can be repeated until at least 3, preferably 10 or more defects are counted, or until 1,000,000 defect opportunities are counted.

9. The ratio of defects/opportunities is calculated. The calculated value is normalized to a Sigma value. The normalization step can be carried out by comparing the ratio to a Sigma chart such as shown as TABLE 1. The TABLE 1 can be stored in the data base of a processor for comparison and identification of Sigma values corresponding to defects per million opportunities (DPMO).

10. Steps 1-9 can be repeated with different values of G₁ . . . G₃, and σ₁ . . . σ₃ to obtain a more accurate determination of the effect of parameters on the Sigma of the system.

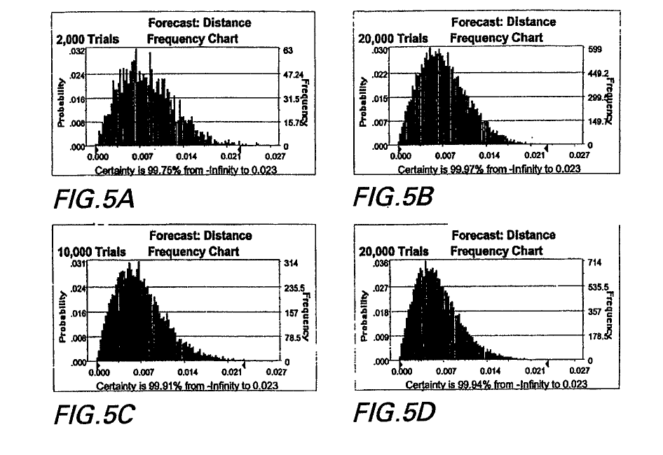

FIGS. 5A through 5D illustrate results of four repetitions of the process 1-9 above with parameters as given in TABLE 2. 2.

TABLE 2

| | | Standard Deviation of | | |
|---|---|---|---|---|
| | Gradient Point | Stocks | DPMO | Sigma |
| (A) | (0.32,0.32,0.36) | .020 | 2500 | 4.25 |
| (B) | (0.32,0.32,0.36) | .0175 | 300 | 4.75 |
| (C) | (0.60,0.20,0.20) | .020 | 900 | 4.5 |
| (D) | (0.60,0.20,0.20) | .0175 | 600 | 4.75 |

FIG. 5A shows results at I equals 0.04, gradient point located (0.32, 0.32, 0.36), stock standard deviation equal to 0.02 and defects equal to 5/2000. FIG. 5B shows results at I equals 0.04, gradient point located (0.32, 0.32, 0.36), stock standard deviation equal to 0.0175 and defects equal to 6/20,000. FIG. 5C shows results at I equals 0.04, gradient point at (0.60, 0.20, 0.20), stock standard deviation equal to 0.02 and defects equal to 9/10,000. FIG. 5D shows results at I equal 0.04, gradient point at (0.60, 0.20, 0.20), stock standard deviation equal to 0.0175 and defects equal to 12/20000. Each histogram is marked by small solid triangle at I/3*√3 so that the data beyond that point in each graph define defects. The defects per million repetitions are calculated from these defects and number of repetitions as DPMO=10ˆ 6*defects/repetitions. The Sigma is derived from a processor database representing TABLE 1.

The foregoing discussion relates to the identification of a step of delivering stock solution to array wells as a CTQ step. Error in delivering individual aliquots will shift individual points in random directions. FIG. 4 shows the specification for the shift of a single point as a circle around that point. Similarly, delivering individual aliquots of stock solution to each mixture in a gradient can be identified as a CTQ area. Again, distance from actual concentration to design concentration for each mixture in the gradient should be no more that I/3*√3. Error in making up a stock solution will shift the entire gradient in the same direction as shown in FIG. 3. The concentration of each component in the mixture is given by equation (III) above and the variance of each component is given by an equation similar to equation (IV), with ²_{Ai} being the variance in the delivery of the amount Aᵢ of stock solution i, etc. The total variance around a given point is found by equation (V) and Sigma is calculated using equations (VI) and (VII). The actual value of Sigma is determined according to delivery accuracy with respect to a composition defined by the gradient point as shown with reference to delivery to array wells.

The following Example is illustrative and should not be construed as a limitation on the scope of the claims unless a limitation is specifically recited.

### EXAMPLE

This example illustrates optimization of a process for the identification of an active and selective catalyst for the production of aromatic carbonates. The process identifies a best cocatalyst from a complex chemical space, where the chemical space is defined as an assemblage of all possible ratios of combinations of certain Group IVb, Group VIb, and Lanthanide Group metal complexes. The chemical space consists of mixtures of levels of the chemical factors of TABLE 3.

TABLE 3

| Group Ivb complex | Group Ib complex | Lanthanide complex |
|---|---|---|
| TiO(acac)2 (A) | Cu(acac)2 (B) | Ce(acac)3 (C) |

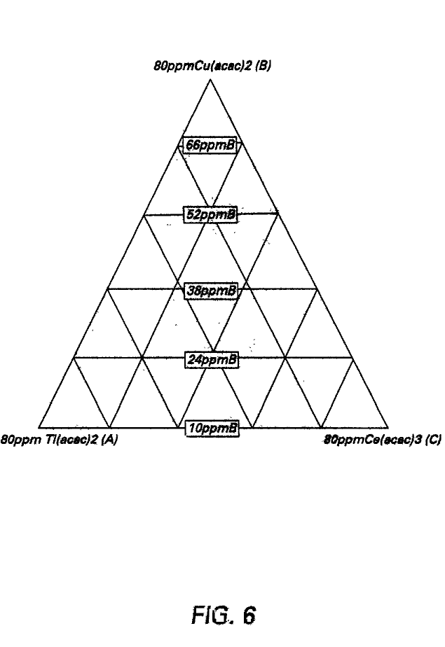

Each of the factors is sampled over a range from 10 to 80 ppm, with a constant total of 100 ppm cocatalyst. FIG. 6 illustrates a sampling of these factor levels according to a ternary gradient experiment. Each line intersection of the FIG. 6 represents one mixture to be tested. FIG. 6 shows 21 mixtures designated as an ABC system. The goal of the program requires that this step have a Sigma value of at least 4.5.

Cocatalyst stock solutions are made up in phenol solvent, each containing 1000 ppm of a level of TABLE 3 cocatalyst. A volume of 0.1 ml. of each cocatalyst solution is added by a dispensing robot to a 1 ml. mixing vial. The dispensing robot has a standard deviation of addition equal to 10% of a volume added in a 0.02 to 0.05 ml. range. Sigma value is then determined using steps 1-9 in the above process as follows in TABLE 4:

TABLE 4

| Steps in process | Example, following the steps |
|---|---|
| A point on a gradient is selected at | (G₁,G₂,G₃) = (38 ppm TiO(acac)2; 38 |
| random. | ppm Cu(acac)2; 24 ppm Ce(acac)3) |
| A design concentration for each stock | S₁ = S₂ = S₃ = 1000 ppm |
| solution (S₁. . . S₃) is selected. | |
| An amount (A₁. . . A₃) of each stock | A₁ = .038 ml, A₂ = .038 ml, A₃ = .024 ml |
| required to generate (G₁,G₂,G₃) is | (from equation (III)) |
| determined | |
| An estimate of the standard deviation for | σ₁ = .0038 ml, σ₂ = .0038 ml σ₃ = .0024 ml |
| the process of addition of each aliquot of | (from robot standard deviation = 10% of |
| stock solution (σ₁. . . σ₃) is made. | the amount added) |
| New values of aliquot amount A′₁. . . A′₃ | A′_{1 =} .0380, A′₂ = .0297, A′₃ = .0226 |
| are randomly selected from normal | |
| distributions having mean A₁. . . A₃ and | |
| standard deviations σ₁. . . σ₃. | |
| Delivered concentrations (G₁′. . . G₃′) of the | G₁′ = 42.06, G₂′ = 32.92, G₃′ = 25.02 |
| components of the mixture resulting from | (calculated by first applying equation III, |
| mixing quantities (A′₁. . . A′₃) of stock | then dividing by ΣG₁′ and multiplying by |
| solutions (S₁. . . S₃) are calculated. | ΣGi′) |
| A distance between delivered and the | Distance = SQRT((42.06 − 38)² + (32.92 − |
| design concentrations of the components | 38)² + (25.02 − 24)²) = 6.586 ppm |
| is calculated | |
| The distances between delivered and | Since I = 14 ppm, I/3 * √3 = 8.08 ppm |
| design concentrations are compared with | |
| the specification of I/3 * √3. | |
| A defect is counted when a distance is | Since 6.586 ppm < 8.08 ppm, no defect. |
| greater than I/3 * √3. | |
| The procedure is repeated until at least 3, | 20 repeats of the process are shown in |
| preferably 10 or more defects are | TABLE 6. One defect (in the third row) is |
| counted, or until 1,000,000 defect | counted A frequency chart of 1000 |
| opportunities are counted. | repeats of the process is shown in FIG. |
| | 7A. |

TABLE 5

| A₁′ | A₂′ | A₃′ | G₁′ | G₂′ | G₃′ | Distance | Defect? |
|---|---|---|---|---|---|---|---|
| 0.0380 | 0.0297 | 0.0226 | 42.06 | 32.92 | 25.02 | 6.586 | No |
| 0.0357 | 0.0400 | 0.0217 | 36.65 | 41.06 | 22.29 | 3.761 | No |
| 0.0443 | 0.0307 | 0.0234 | 45.03 | 31.17 | 23.80 | 9.803 | Yes |
| 0.0404 | 0.0334 | 0.0204 | 42.86 | 35.49 | 21.65 | 5.959 | No |
| 0.0315 | 0.0415 | 0.0243 | 32.35 | 42.62 | 25.03 | 7.375 | No |
| 0.0374 | 0.0440 | 0.0267 | 34.60 | 40.69 | 24.70 | 4.390 | No |
| 0.0395 | 0.0343 | 0.0281 | 38.79 | 33.64 | 27.57 | 5.689 | No |
| 0.0374 | 0.0407 | 0.0241 | 36.58 | 39.86 | 23.57 | 2.377 | No |
| 0.0391 | 0.0378 | 0.0212 | 39.82 | 38.53 | 21.64 | 3.029 | No |
| 0.0365 | 0.0434 | 0.0234 | 35.33 | 42.04 | 22.63 | 5.038 | No |
| 0.0323 | 0.0384 | 0.0239 | 34.12 | 40.58 | 25.30 | 4.836 | No |
| 0.0382 | 0.0360 | 0.0205 | 40.31 | 38.03 | 21.66 | 3.290 | No |
| 0.0364 | 0.0391 | 0.0237 | 36.70 | 39.44 | 23.87 | 1.945 | No |
| 0.0332 | 0.0395 | 0.0249 | 34.02 | 40.46 | 25.52 | 4.925 | No |
| 0.0332 | 0.0312 | 0.0236 | 37.73 | 35.45 | 26.82 | 3.811 | No |
| 0.0385 | 0.0370 | 0.0224 | 39.31 | 37.77 | 22.92 | 1.710 | No |
| 0.0395 | 0.0343 | 0.0260 | 39.59 | 34.34 | 26.07 | 4.497 | No |
| 0.0380 | 0.0406 | 0.0255 | 36.50 | 38.98 | 24.52 | 1.866 | No |
| 0.0397 | 0.0356 | 0.0243 | 39.81 | 35.77 | 24.42 | 2.902 | No |

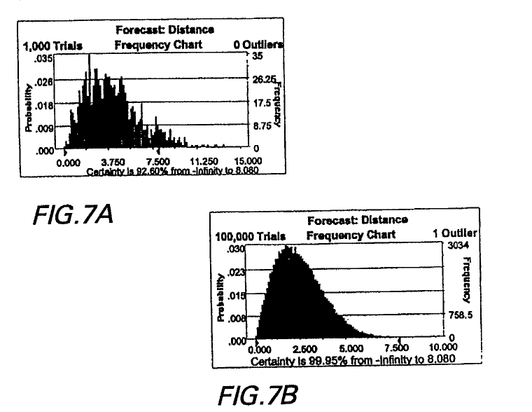

The FIG. 7A graph shows that 7.4% (74,000 DPMO) of the repetitions of the calculations is off spec (distance greater than 8.08). A DPMO of 74,000 gives a Sigma value less than 3.0 according to Table 1. Accordingly, the results identify the robotic step as a critical area of low quality.

Six Sigma analysis methods are used to determine potential root causes of the excessive variability of the robot. Potential root causes include the viscosity of the stock solution; the speed of withdrawal of the aliquot of stock solution from the stock solution vial; the speed of addition of the aliquot to the sample vial; and the diameter of the pipet tip. Six Sigma improvement methods are used to find that a critical interaction occurs between viscosity of the stock solution and the speed of withdrawal. At high withdrawal rates and low viscosity solutions, variability is high because of bubble formation in the pipet tip. Adjusting sample viscosity and lowering withdrawal rate decreases variability.

The changes result in a decreased standard deviation of the dispensing robot additions to a constant 0.0020 ml over the 0.02 to 0.05 ml. range. The process of steps 1-9 was then repeated to provide the results shown in FIG. 7B. The FIG. 7B graph shows that 0.05% (500 DPMO) of the simulation is off spec (distance greater than 8.08), giving a Sigma value better than 4.75.

While preferred embodiments of the invention have been described, the present invention is capable of variation and modification and therefore should not be limited to the precise details of the EXAMPLE. For example, the mixture of interest and the subject of the invention may be a quaternary or pentanary or other multi-component mixture. The invention includes changes and alterations that fall within the purview of the following claims.

1. A method to improve a CHTS experiment, comprising steps of:
formulating an array of a mixture of at least two components;
conducting an experiment on the array to produce results; and
applying at least one Six Sigma technique to a step of the experiment to improve results of the experiment.

2. The method of claim 1, additionally comprising monitoring the step of formulating the array of a mixture.

3. The method of claim 1, additionally comprising monitoring the step of formulating the array of a mixture and identifying the step as an opportunity for a defect.

4. The method of claim 1, wherein the array of a mixture is formulated by delivering components of reactants to a well of an array plate.

5. The method of claim 1, wherein the array of a mixture is formulated by delivering components of reactants to a well of an array plate using a robotic dispenser.

6. The method of claim 1, wherein applying at least one Six Sigma technique includes identifying a defect opportunity.

7. The method of claim 1, wherein applying at least one Six Sigma technique includes identifying a defect opportunity selected from steps of monitoring a stock precursor solution, mixing an aliquot of the stock solution with an aliquot of another solution, delivering a mixture of the aliquots to a well of an array plate, effecting a condition of reaction on the mixture, detecting a result of the reaction and analyzing the result to determine either a lead or to determine a candidate library for reiterating the experiment.

8. The method of claim 1, comprising (A) an iteration of steps of (i) formulating an array of mixtures of at least two components; (ii) reacting the array mixtures; and (iii) evaluating a set of products of the reacting step and (B) repeating the iteration of steps (i), (ii) and (iii) wherein components of a successive array of mixtures selected for a step (i) are chosen as a result of an evaluating step (iii) of a preceding iteration.

9. The method of claim 1, wherein applying the Six Sigma technique comprises determining a Sigma value as equal to an absolute value function of a difference between average value of measurements on the system minus a nearest specification divided by a standard deviation of the measurements on the system.

10. The method of claim 1, wherein applying the Six Sigma technique comprises determining a Sigma value as equal to per million occurrences of a ratio of number of defects of a product to number of opportunities for defect times number of units of the product.

11. The method of claim 1, wherein applying at least one Six Sigma technique includes identifying a step of delivering stock solution to a well or substrate as a critical to quality defect opportunity.

12. The method of claim 1, wherein applying at least one Six Sigma technique includes identifying a defect as a ternary mixture concentration that deviates more than (I/3*√3) from a design concentration where I is a height of an equilateral triangle of a graphic representation of the mixture.

13. The method of claim 1, wherein applying at least one Six Sigma technique includes calculating a Sigma value equal to (I/3*√3)/_{P} where I is a height of an equilateral triangle of a graphic representation of the mixture and _{P} is standard deviation.

14. The method of claim 1, wherein the Six Sigma technique includes establishing a project goal Sigma value of at least 4.5.

15. The method of claim 1, wherein the Six Sigma technique includes establishing a project goal Sigma value of at least 5.0.

16. The method of claim 1, wherein the Six Sigma technique includes establishing a project goal Sigma value of at least 5.5.

17. The method of claim 1, wherein the Six Sigma technique includes (1) selecting a point on a gradient representation of the mixture; (2) selecting a design concentration for each stock solution and an estimate of the standard deviation for each stock solution used to generate a mixture represented by the point; (3) determining an amount of each stock solution required to generate the mixture; (4) randomly selecting another stock concentration value from normal value distributions of concentration from the point mixture; (5) calculating a delivered concentration of components of a mixture resulting from mixing design amounts of stock solution; (6) calculating a distance between delivered concentration and the design concentration; and (7) counting a defect when the calculated distance exceeds I/3*√3.

18. The method of claim 17, wherein steps (3) to (7) are repeated until at least 3 defects are counted.

19. The method of claim 17, wherein steps (3) to (7) are repeated until at least 10 defects are counted.

20. The method of claim 17, wherein steps (3) to (7) are repeated until 1,000,000 defect opportunities are counted.

21. The method of claim 17, wherein the mixture is a ternary, quaternary or pentanary mixture.

22. The method of claim 17, wherein steps (3) and (4) are determined according to formulas (III) through (VII) based on an assumption of no error in the stock solution concentration.

23. The method of claim 17, wherein the distance between delivered concentration and design concentration is calculated according to the formula (where SQRT is the square root function).

24. The method of claim 1, wherein the Six Sigma technique identifies at least one of viscosity of stock solution, speed of withdrawal of solution from a stock solution vial, speed of addition to an array well and diameter of pipet tip as an area for improving Sigma of the formulating step.

25. The method of claim 1, wherein the Six Sigma technique includes calculating a ratio of defects/opportunities.

26. The method of claim 1, wherein the Six Sigma technique includes calculating a ratio of defects/opportunities and the calculated ratio is normalized to a Sigma value.

27. The method of claim 1, wherein the Six Sigma technique includes calculating a ratio of defects/opportunities and the calculated ratio is normalized to a Sigma value by comparing the ratio to a Sigma chart.

28. The method of claim 1, wherein the Six Sigma technique includes calculating a ratio of defects/opportunities and the calculated ratio is normalized to a Sigma value by comparing the ratio to a Sigma chart stored in the data base of a processor.

29. The method of claim 1, wherein the Six Sigma technique includes calculating a ratio of defects/opportunities and the calculated ratio is normalized to a Sigma value corresponding to defects per million opportunities (DPMO).

30. The method of claim 1, wherein a low Sigma cause is identified and Sigma is improved by improving the low sigma cause.

31. The method of claim 1, wherein the components include a catalyst system comprising combinations of Group IVB, Group VIB and Lanthanide Group metal complexes.

32. The method of claim 1, wherein the components include a catalyst system comprising a Group VIII B metal.

33. The method of claim 1, wherein the components include a catalyst system comprising palladium.

34. The method of claim 1, wherein the components include a catalyst system comprising a halide composition.

35. The method of claim 1, wherein the components include an inorganic co-catalyst.

36. The method of claim 1, wherein the components include a catalyst system that includes a combination of inorganic co-catalysts.

37. A method, comprising:
identifying a reactant delivering step as an opportunity for a defect in a CHTS experiment;
measuring a number of units produced by the delivering step;
measuring defects in the units produced by the delivering step of the repeated CHTS; and
calculating a defects per unit for the delivering step.

38. The method of claim 37, wherein the CHTS experiment comprises an iteration of steps of simultaneously reacting a multiplicity of tagged reactants and identifying a multiplicity of tagged products of the reaction and evaluating products after completion of a single or repeated iteration.

39. The method of claim 37, wherein the CHTS experiment comprises effecting parallel chemical reactions of an array of reactant mixtures.

40. The method of claim 37, wherein the CHTS experiment is characterized by parallel reactions at a micro scale.

41. The method of claim 37, wherein the CHTS experiment comprises (A) an iteration of steps of (i) delivering mixtures of reactants to array wells; (ii) reacting the mixtures and (iii) evaluating a set of products of the reacting step and (B) repeating the iteration of steps (i), (ii) and (iii) wherein a successive mixture of reactants selected for a step (i) is chosen as a result of an evaluating step (iii) of a preceding iteration.

42. The method of claim 37, wherein the CHTS experiment comprises effecting parallel chemical reactions of an array of ternary reactant mixtures.

43. A method, comprising:
identifying a reactant delivering step or stock formulating step as an opportunity for a defect in a mixture experiment;
measuring a number of units produced by the delivering step or formulating step;
measuring defects in the units produced by the delivering step or the formulating step; and
calculating a defects per unit for the delivering step or formulating step.

44. The method of claim 43, wherein the experiment is a ternary, quaternary or pentanary mixture experiment.

45. The method of claim 43, wherein the experiment is a ternary, mixture experiment.
